(19) United States
(12) Patent Application Publication
(10) Pub. No.: US 2026/0305589 A1
(43) Pub. Date: Oct. 8, 2026

(54) DEVICE AND METHOD FOR UTILIZING FOOD WASTE

(71) Applicants: SmartBreed AG, Zufikon (CH); SmartBreed AG, Zufikon (CH)

(72) Inventors: Patrik Bertschi, Dietikon (CH); Christoph Bertschi, Dietikon (CH)

(21) Appl. No.: 19/469,022

(22) PCT Filed: Feb. 16, 2024
(86) PCT No.: PCT/EP2024/053941

§ 371 Date: Sep. 25, 2025

(30) Foreign Application Priority Data
Mar. 23, 2023 (DE) 10 2023 202 653.8

(51) Int. Cl. A01K 67/362; A01K 67/364

(52) U.S. Cl. CPC A01K 67/362; A01K 67/364

(57) ABSTRACT

The invention relates to a device for growing insect larvae with a breeding container, wherein pressure reduction elements are present in the breeding container and at least one air supply pipe at the bottom of the breeding container, via which air can be pumped into the breeding container. The invention also relates to a method for growing insect larvae with such a device, wherein first the breeding container of the device is filled with feed for the insect larvae to less than 10% and subsequently insect larvae are added.

### CROSS REFERENCE TO RELATED APPLICATIONS

This application is a US National Phase of PCT/EP2024/053941 filed on Feb. 16, 2024, which claims priority to DE 10 2023 202 653.8 filed on Mar. 23, 2023, the entire disclosures of which are hereby incorporated by reference and relied upon.

### BACKGROUND OF THE INVENTION

Field of the Invention. The invention relates to a device and a method for rearing insect larvae.

Description of Related Art. Larvae of insects such as, for example, insect larvae of the black soldier fly can utilize moist food. Insect larvae of insects can grow, for example, with the help of vegetables, pasta, meat or feces. Therefore, insect larvae can be fed with the help of food waste in order to subsequently use grown insect larvae, for example, as feed for animals.

### BRIEF SUMMARY OF THE INVENTION

It is an object of the invention to breed insect larvae efficiently.

The object can be solved by a device with the features of the first claim as well as by a method with the features of the independent claim. The dependent claims relate to advantageous embodiments.

A device is configured to solve the object for growing insect larvae. The device is particularly configured such that insect larvae can be bred with the help of biomasses such as food waste. In particular, the device is configured such that wet feed for the insect larvae, which has a high water content of, for example, at least 40 wt. % or at least 50 wt. %, can be utilized. It can be a sludge-like feed mass that can be processed by the device.

For example, food waste with a water content of at least 40 wt. % or at least 50 wt. % can be utilized for rearing the insect larvae. The water content can be, for example, approximately 60 wt. %.

The device can comprise a breeding container in which insect larvae can grow.

The device can be configured such that first feed, for example in the form of wet food waste, and then insect larvae can be brought into the breeding container, preferably automated with the help of an electronic control device. The insect larvae that are brought into the breeding container can be, for example, already three, four, five, six or seven days old. The insect larvae can feed on the feed and thus grow. When the insect larvae have reached a desired size, the insect larvae can be harvested and used, among other things, as animal feed. The insect larvae can be, for example, 8 to 12 days old at the time of harvest.

Insect larvae of black soldier flies are preferably used as insect larvae because these insect larvae can particularly efficiently utilize moist or wet biomass, such as food waste. Food waste can comprise, for example, vegetable waste and/or pasta waste.

The device can be configured such that first wet feed can be brought into the breeding container until the breeding container is partially filled. Then insect larvae can be added. At a later time, additional wet feed can be added depending on demand. Wet feed can be added periodically and repeatedly. For example, new wet feed can be added daily. The daily newly added feed amount can be increased daily to meet the growing feed demand of the growing insect larvae. The device can be configured to automatically bring feed and/or insect larvae into the breeding container in this manner. A time interval between two feedings can be selected such that at least 70% or at least 80% or at least 90% of the feed has been utilized before new feed is added.

When the insect larvae have reached a desired predetermined size, the addition of further feed can be stopped. It can then be waited until the contents of a breeding container are no longer wet and consequently dry. It can thus be achieved that the feed is practically completely utilized. Furthermore, the further processing of the contents of the breeding container can be facilitated in this way. To achieve this, for example, on the last day before harvesting the insect larvae, the supply of wet feed can be stopped. On the following day, harvesting can then take place. The device can be configured to automatically stop the periodic supply of wet feed.

For example, the usable volume of the breeding container available for filling can be filled 5% to 15% with wet feed, for example automatically. The height of the wet feed in the container can initially be limited to, for example, a maximum of 15 cm or a maximum of 10 cm or a maximum of 5 cm. However, the bottom can be completely covered by the wet feed after the initial supply. Subsequently, insect larvae can be added, for example automatically. When the insect larvae have largely utilized the wet food waste, wet food waste is added again. Depending on demand, wet food waste can thus be added step by step. The initially still predominantly empty breeding container can thus be filled step by step. The usable volume of the breeding container can thus have been filled, for example, to at least 80% or to at least 90% by the time the insect larvae are removed. By usable volume or usable volume is meant the volume of the container that is maximally available for filling with flowable feed. For example, the volume above feed supply nozzles does not belong to the usable volume.

By gradually adding wet feed, it is avoided that water can accumulate in the bottom such that insect larvae can drown in it. Also, a very good efficiency can be achieved in this way.

The breeding container can have one or more inlet openings and/or one or more pipes for supplying feed at its top side, through which moist food waste and/or insect larvae can be brought into the breeding container. The one or more inlet openings and/or the one or more feed supply pipes can be closable. Thus, one or more closures, for example covers made of metal, can be present with which, for example, the one or more openings can be closed. The one or more feed supply pipes can comprise valves for closing.

A breeding container can be a module that is at least predominantly closed on its underside and that is at least predominantly open on its top side. By module is meant a structural unit that has been manufactured separately from other parts of the device. A module can comprise one or more pipes for the supply of feed. A module of the device can have been placed on another module of the device. The device then comprises two breeding containers. The bottom of the upper breeding container can then close the top side of the lower breeding container. For the upper breeding container, a separate cover can be present to also close the top side of the uppermost breeding container and thus protect it from external influences.

The device can comprise several breeding containers to be able to breed insect larvae in parallel. The age of insect larvae in a first breeding container can differ from the age of insect larvae in a second breeding container. The age of insect larvae in a third breeding container can differ from the age of the insect larvae from the first and second breeding containers. Insect larvae can thus be harvested at different times. A quasi-continuous production of insect larvae is thus possible.

The breeding containers can be arranged next to each other and/or behind each other. The device can comprise, for example, 4 to 20 breeding containers.

The device can comprise a pump or a compressor to be able to pump wet feed such as, for example, wet food waste into a breeding container. A side channel compressor can be provided as a compressor. The wet food waste can be so viscous or sludge-like that it can be pumped into a breeding container with a pump or a compressor. Wet feed can be pumped into a breeding container via the aforementioned one or more pipes. The water supply device can comprise an electronic control device with which the pumping is controlled.

The device can comprise a water supply device with which water can be added to the feed before the feed is brought into the breeding container, for example pumped into the breeding container with the help of the pump. The water supply device can comprise an electronic control device with which the supply of water to the feed can be controlled, for example depending on the feed volume and/or depending on a sensor with which the water content in the feed can be determined.

The device can comprise a feed station in which feed and water can be mixed to provide wet feed. From the feed station, the wet feed can be brought into the breeding container. It can be a feed station as provided for pig fattening to feed pigs with liquid feed.

The device can comprise a removal device. The contents of a breeding container can be removed by the removal device. Towards the end of rearing, the contents can consist largely or even practically completely of insect larvae and feces. With the help of the removal device, insect larvae and feces can then be removed from the breeding container. The removal device can be configured such that it can automatically remove the contents such as insect larvae and feces from the breeding container. By removing insect larvae and feces, the breeding container can be emptied to be able to harvest insect larvae that have reached a desired size. This can be the case, for example, in the case of insect larvae of the black soldier fly 6 to 10 days after the start of breeding.

The breeding container can have one or more outlet openings at its underside through which insect larvae and feces can be removed from the breeding container. An outlet opening can be closed, for example, by a closure such that the closure must be opened suitably wide for removal. For suitable closing of the one or more outlet openings, one or more closures such as, for example, covers made of metal can be provided. The one or more outlet openings at the underside are suitably widely closed when this prevents wet feed from falling or flowing out of the breeding container downward.

The removal device can comprise one or more screw conveyors. The one or more screw conveyors can be arranged adjacent to the bottom of a breeding container. The one or more screw conveyors can be configured such that they can convey the contents of a breeding container to an outlet opening. The provision of screw conveyors is particularly preferable for space reasons, since the usable volume of a breeding container can thus be optimized. The one or more screw conveyors can be part of the mentioned module.

The contents that are conveyed by screw conveyors for the purpose of removal ideally consist at least practically completely of grown insect larvae and feces. Furthermore, the contents are ideally dry, i.e., no longer a sludge-like mass.

A breeding container can comprise at least 3 screw conveyors or at least 5 screw conveyors or at least 8 screw conveyors. A breeding container can comprise no more than 20 screw conveyors or no more than 15 screw conveyors or no more than 12 screw conveyors.

A screw conveyor can be coupled at one end to an adjacent screw conveyor such that the screw conveyors can only rotate together. One drive is then sufficient to be able to rotate all screw conveyors. Two adjacent screw conveyors can be coupled to each other by gears and/or one or more chains and/or belts.

The removal device can comprise one or more pushers that can be arranged at the bottom of the breeding container. With the help of the pushers, the contents of a breeding container can be pushed to one or more outlet openings. However, pushers are less suitable compared to screw conveyors.

The removal device can include a bottom that is inclined toward the outlet opening.

An outlet opening can be located in or at a side wall and/or in the bottom, i.e., in the floor, of a breeding container. Via the outlet opening, the contents can reach a removal shaft. The outlet opening can open into a ramp or be provided at the end of a ramp. The lower end of the ramp can open into the removal shaft to thus be able to transport the contents from the outlet opening to the removal shaft by means of gravity. Via the removal shaft, the contents can be removed at the lower end. The removal shaft can be part of the mentioned module. The removal shaft can be arranged on the outside of a side wall of the breeding container.

One or more outlet openings can be closed, for example, by one or more closures, for example by one or more sliders. A drive for the one or more closures can be arranged on the underside of the bottom of the breeding container. Closures and/or drive can be part of the module. A closure can be arranged on the top side of the mentioned ramp. The contents of a breeding container can then slide down the ramp only after opening the closure.

The one or more closures can close the one or more outlet openings such that no feed can flow through a closed outlet opening. However, a small gap can be present through which water can drain. The gap can be, for example, no wider than 5 mm or no wider than 3 mm or no wider than 1 mm. The width of the gap can be at least 0.4 mm. This better prevents water from accumulating in the bottom of a breeding container in which insect larvae could drown.

For example, at the underside of a shaft, a separation device can be provided by which insect larvae can be separated from feces. A sieve can be provided as a separation device or the separation device can comprise a sieve. Insect larvae can be separated from feces by the sieve.

A breeding container can comprise a flat bottom or at least substantially flat bottom that adjoins a slope on one side. The slope can bend obliquely downward toward an outer wall of the breeding container. The bend or slope can form the mentioned ramp. The top side of the breeding container can run like the underside of the breeding container. The top side then also has a flat course that runs obliquely downward toward the mentioned outer wall. It is then unproblematically possible to be able to place or stack two breeding containers designed as modules on top of each other.

The removal device can comprise other means for removal such as, for example, a plurality of inclined channels or nozzles via which the contents can be led out of the breeding container. Viewed from above, each channel or each nozzle can be arranged between pressure reduction elements. An opening via which the contents can leave the removal device can be provided with a cover.

Within a breeding container, one or more pressure reduction elements can be present with which it is prevented that an excessively high pressure is exerted on insect larvae. A pressure reduction element is a component that is installed in the breeding container to regulate and stabilize the pressure within a filled breeding container. A pressure reduction element serves to avoid damage to the insect larvae. The one or more pressure reduction elements can be part of the mentioned module.

A pressure reduction element can be an elongated component whose ends can be attached to two opposite side walls of the breeding container. A pressure reduction element can be positioned obliquely within the breeding container such that feed can slide or flow down from the pressure reduction element due to gravity. A pressure reduction element can be a strip. A pressure reduction element can be shaped in cross-section like a V rotated 180° or a U rotated 180°. A pressure reduction element can run horizontally within the breeding container. A pressure reduction element can be a perforated component. The holes can be adapted to the size of insect larvae such that insect larvae can migrate through the holes to be able to reach feed as quickly as possible. Excessively large holes are to be avoided so that pressure can be suitably reduced. The maximum size of the holes can thus be adapted to the maximum size of insect larvae. The holes can be, for example, a maximum of 10 mm wide or a maximum of 5 mm wide. The holes can be, for example, a maximum of 30 mm long or a maximum of 20 mm long or a maximum of 10 mm long or a maximum of 5 mm long. The holes can be, for example, a minimum of 10 mm wide or a minimum of 5 mm wide and/or long. The holes are preferably circular. However, they can also be elongated holes.

Pressure reduction elements can be arranged above each other and/or next to each other within a breeding container to be able to suitably reduce pressure. Pressure reduction elements that are arranged above each other can be arranged overlapping when viewed from above to be able to suitably uniformly reduce pressure.

Pressure reduction elements can be arranged within a breeding container such that one or more shafts remain through which feed can fall unhindered. The one or more shafts can run exactly vertically. Feed can then reliably reach the bottom of a breeding container, which is advantageous for rearing insect larvae. In particular, it is possible that feed can quickly fill the space below pressure reduction elements by flowing, which can have a favorable effect on the growth of insect larvae. One or more inlet openings or pipe openings through which feed can and should be brought into the breeding container can be arranged at the top side of the breeding container exactly above the one or more shafts to enhance this effect.

Preferably, pressure reduction elements are arranged in a first plane and pressure reduction elements in a second plane and pressure reduction elements in a third plane, etc. The planes can be arranged parallel to each other. The distances between two adjacent planes can be equal. The distances between two adjacent pressure reduction elements of a plane can be equal. It is thus possible to uniformly protect insect larvae in the breeding container from excessive pressure.

Preferably, the pressure reduction elements of one plane are arranged offset relative to pressure reduction elements of a plane below and/or above. This arrangement has proven to be favorable for counteracting excessively high pressures. Pressure reduction elements of two planes between which a plane with pressure reduction elements is located can be arranged not offset relative to each other.

A pressure reduction element can be a maximum of 4 m or a maximum of 3 m long. The maximum width of a pressure reduction element can be 1000 mm. The minimum width of a pressure reduction element can be 10 mm. The maximum height of a pressure reduction element can be 1000 mm. The minimum height of a pressure reduction element can be 10 mm. The maximum thickness of each wall of the pressure reduction element can be at least 20 mm. The minimum thickness of each wall of the pressure reduction element can be at least 0.5 mm.

The device can comprise an evaporation device for cooling with which liquid present in the breeding container can be evaporated in a controlled manner when feed and insect larvae are in the breeding container. The evaporation device is configured such that cooling can be achieved by evaporation, i.e., a temperature can be maintained or lowered. The evaporation device can be configured such that cooling to a desired preset temperature is performed when needed, preferably automatically. The desired preset temperature can be at least 27° C. or at least 33° C. The desired preset temperature can be less than 40° C. or less than 37° C.

The evaporation device can be configured such that evaporated liquid can be led upward out of the breeding container. The evaporation device can be configured such that the degree of evaporation and thus the degree of cooling can be controlled.

Evaporation by an air stream is to be preferred. The evaporation device then comprises a device for generating an air stream that is led through the breeding container. The device for generating an air stream can comprise a pump, a blower, a compressor and/or a fan. The evaporation device can prevent insect larvae from excessively heating the substrate and thus endangering the rearing success. Furthermore, in the case of an air stream, gases such as CO2 can be reliably and sufficiently completely transported out of the breeding container. The breeding container can thus be degassed by an air stream.

The breeding container can comprise one or more air outlet openings at its top side. A blower or a fan can be adjacent to each air outlet opening to blow air outward through the air outlet opening. An air outlet opening can be present in a side wall of the breeding container. An associated fan can be mounted on the inside of the side wall. The one or more blowers or fans can be part of the module. By providing one or more fans, it is avoided that a disadvantageous humid space can form above the mixture consisting of insect larvae, feed and/or feces.

Compressor, pump, blower and fan differ in their performance. A compressor can pump air at higher pressure than a pump, a blower or a fan can. A pump can pump air at higher pressure than a blower or a fan can. A blower can pump air at higher pressure than a fan can.

An air outlet opening can be covered by louvers to prevent the penetration of contaminants. Slots can remain between the louvers through which air can exit. The louvers can be arranged overlapping to provide particularly good protection against contaminants. The louvers can be part of the module.

At the bottom of the breeding container, one or more air supplies can be present through which air can enter the breeding container. These can be one or more air supply pipes. The one or more air supply pipes can be arranged above one or more screw conveyors. The one or more air supply pipes can be arranged adjacent to one or more pressure reduction elements, preferably to the lowest pressure reduction elements. The one or more air supply pipes can be arranged adjacent to the underside of one or more pressure reduction elements. The one or more pressure reduction elements of a breeding container can be arranged above the one or more air supply pipes.

The one or more air supply pipes can be porous so that air can exit particularly uniformly distributed over the surface area of the one or more air supply pipes. The one or more air supply pipes can have been manufactured by pressing spheres to obtain a porous surface. The one or more air supply pipes can consist of metal. The one or more air supply pipes can be part of the module. This advantageously achieves uniform oxygen supply for the insect larvae and/or uniform drying and/or uniform loosening of the contents of the breeding container and/or uniform cooling of the larvae through evaporation.

Preferably, air is only supplied into the breeding container adjacent to the underside and/or only led out of the breeding container adjacent to the top side of the breeding container.

It is also possible that the main purpose of an air stream that is led, for example, by a pump from an air inlet opening through the breeding container to an air outlet opening consists in degassing.

With small insect larvae, it may be necessary to supply heat, for example by supplying heated air or by supplying heat via a heat exchanger arranged in the breeding container. A control device of the device can be configured such that it controls heat supply depending on the size of insect larvae.

Alternatively or additionally, the control device can control the air supply and thus the cooling by evaporative cooling to a breeding container of the device. This can be done depending on a determined cooling requirement.

When air is led through the breeding container, this is usually done without interruption during rearing to continuously remove, for example, gases from the breeding container. However, ventilation can also occur at intervals of a few seconds to a few minutes. Especially when the larvae are still small, this can be advantageous so that the larvae feel as undisturbed as possible and the substrate is not moved too much. When the larvae have grown larger, it is preferably switched to continuous ventilation.

The device can comprise a plurality of breeding containers. The device can comprise a heat exchanger device with which heat can be exchanged between breeding containers. If there are a plurality of breeding containers, small insect larvae can be in a first breeding container and large insect larvae in a second breeding container. It may be that the first breeding container with the small insect larvae must be heated and the second breeding container with the large insect larvae must be cooled. Through the heat exchanger, heat can then be exchanged between the first and second breeding containers such that the first breeding container is thereby heated and the second breeding container is thereby cooled. The energy requirement needed for rearing can thus be kept low. In principle, two breeding containers between which heat is exchanged are arranged next to each other.

A breeding container can comprise a cleaning device to be able to clean the breeding container with water. A breeding container can comprise a spray device to be able to spray water for cleaning. The spray device can comprise a spray ball through which water can be sprayed in many different directions. It has been found that through such non-sterile cleaning with water, preferably with sprayed water, beneficial bacteria can remain in the breeding container that can promote the rearing of insect larvae. The cleaning device can be part of the module.

A breeding container can be a maximum of 2 m or a maximum of 1.5 m or a maximum of 1 m high to avoid the risk of insect larvae drowning when using wet feed. A breeding container can be at least 0.5 m or at least 0.8 m high to be able to maximize usable volume. A breeding container can be, for example, at least 1 m wide and/or at least 1 m deep. A breeding container can be, for example, at least 2 m wide and/or at least 2 m deep. A breeding container can be, for example, a maximum of 4 m wide and/or a maximum of 4 m deep. A breeding container can be, for example, a maximum of 3 m wide and/or a maximum of 3 m deep.

With a breeding container that is 2 m×2 m×1 m in size, approximately 2.6 tons of insect larvae can be produced per month.

A breeding container can consist entirely or partially of metal, for example of corrosion-resistant steel. A breeding container can consist entirely or partially of plastic. Pressure reduction elements in the breeding container can consist completely or partially of metal or plastic.

A breeding container can comprise one or more temperature sensors. The one or more temperature sensors can be used to control the temperature control of the breeding container.

The invention also relates to a method for rearing insect larvae with a previously described device.

Additives such as minerals and/or amino acids can be added to the feed.

The device can comprise a biogas plant that can generate a gas such as methane from the feces of the insect larvae. It has been found that a high efficiency for generating gas from biomass can thus be achieved. Under certain circumstances, the efficiency can even be greater compared to the case where gas is generated directly from biomass such as food waste in a biogas plant. A biogas plant is a facility that can convert organic waste such as manure, corn or other plant residues into biogas. Waste can be fermented in a closed container (fermenter) of the biogas plant under anaerobic conditions (without oxygen), whereby biogas is released from the organic substances. Biogas consists mainly of methane (CH4) and carbon dioxide (CO2) and can be used as fuel for electricity and heat generation.

The device can be configured such that the feces of the insect larvae separated from the insect larvae can be fed to the biogas plant. In the biogas plant, biogas can be generated from the feces in the biogas plant.

The device can comprise a biodiesel plant that can generate biodiesel from the fat of the insect larvae. Biodiesel is a fuel that can be produced from animal fats. Unlike fossil fuels such as gasoline or diesel, which are obtained from crude oil, biodiesel is a renewable fuel that comes from natural sources such as animal fats. Biodiesel has higher combustibility and lower pollutant emissions compared to conventional diesel, making biodiesel a more environmentally friendly alternative. A biodiesel plant is a facility in which biodiesel can be produced. Biodiesel can be obtained from vegetable oils or animal fats that react in a chemical process with alcohol and a catalyst to form a compound called biodiesel.

The device can comprise a separation device for insect larvae to separate fat of the insect larvae from other components of the insect larvae. The separation device for insect larvae can comprise a press with which insect larvae can be pressed to separate fat of the insect larvae from other components of the insect larvae. The fat of the larvae can be used, for example, for or as animal feed, for or as food, in pharmaceutical products or in cosmetic products.

The device can comprise a fat supply device with which the separated fat can be conveyed, for example automatically, to the biodiesel plant. The other components can be used, for example, as animal feed.

Through the invention, waste can thus be converted into combustible gas, liquid fuel and animal feed with good efficiency and low technical effort.

### BRIEF DESCRIPTION OF THE SEVERAL VIEWS OF THE DRAWINGS

The figures show:

FIG. 1 is cross-section through a breeding container;

FIG. 2 is a side view of the breeding container from FIG. 1;

FIG. 3 is a pressure reduction element; and

FIG. 4 is a device with two stacked breeding containers

### DETAILED DESCRIPTION OF THE INVENTION

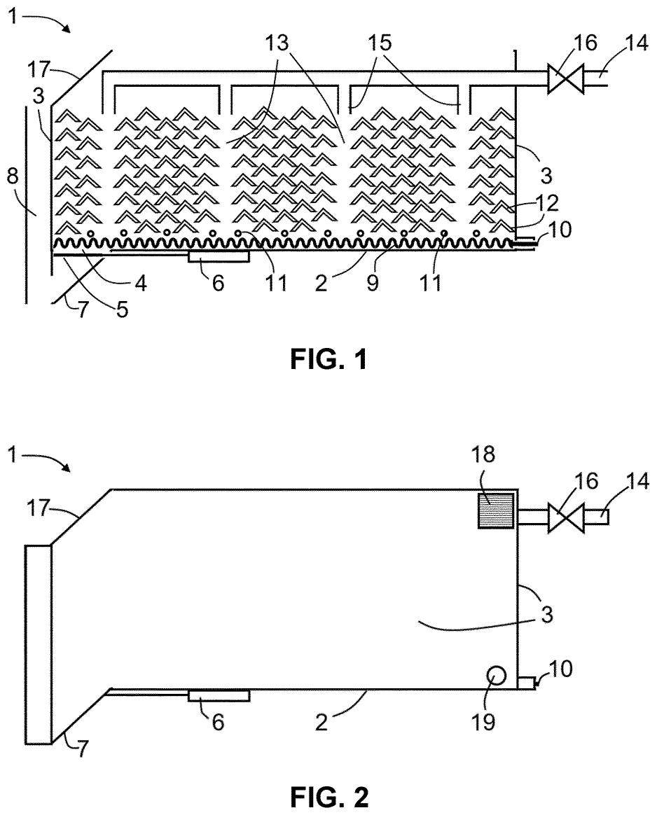

FIG. 1 shows in cross-section a breeding container 1 that can have been manufactured separately from other parts as a structural unit to form a module. The breeding container 1 can comprise a bottom 2 and surrounding side walls 3. In the bottom 2, an outlet opening 4 can be present adjacent to a side wall 3. The outlet opening 4 can extend over the entire depth or almost over the entire depth of the breeding container 1. Alternatively, a plurality of outlet openings 4 can be present that are arranged next to each other to thus cover the entire depth. Each outlet opening 4 can be closed with a slider 5. For example, an electric drive 6 can be mounted on the underside of the bottom 2 with which the slider or sliders 5 can be moved back and forth between an opening position and a closing position. In the opening position of the slider(s) 5, contents can fall from the breeding container 1 through the one or more outlet openings 4 onto a ramp 7 and subsequently slide along the ramp 7 toward a removal shaft 8. The ramp 7 can extend over the entire depth or almost over the entire depth of the breeding container 1. Alternatively, a plurality of ramps 7 can be present that are arranged next to each other to thus cover the entire depth. There can be, for example, five to fifteen, for example ten, screw conveyors 9 next to each other. Contents that have slid down can be removed via the removal shaft 8. The removal shaft 8 is particularly useful when an identical breeding container is placed on the breeding container 1. Then the contents of the identical breeding container can be led out via the removal shaft 8 of the breeding container located below.

Adjacent to the bottom 2, there can be one or more screw conveyors 9 with which contents in the breeding container 1 can be conveyed toward the outlet opening 4. A plurality of screw conveyors 9 are to be preferred to cover the entire depth of the breeding container 1. Each screw conveyor 9 can be located in an upwardly open channel to be able to convey contents of the breeding container along the channel to the outlet opening 4. At one end of each screw conveyor 9, an outlet opening 4 can be present. The other opposite end of each screw conveyor 9 can be coupled to a drive. Each screw conveyor 9 can extend from one side wall 3 to an opposite side wall 3 and thus over the entire width of the breeding container 1. A drive shaft 10 of each screw conveyor 9 can pass through a side wall 3. The drive shaft 10 can be connected to a drive, for example an electric drive. Between adjacent screw conveyors 9, there can be equal distances to be able to convey uniformly.

Above the one or more screw conveyors 9, one or more pipes 11 for the supply of air can be arranged. The jackets of the one or more air supply pipes 11 can be porous. Air can be pumped into the breeding container 1 via the one or more air supply pipes 11. Each air supply pipe 11 can extend over the entire depth or almost over the entire depth of the breeding container 1. One end of each air supply pipe 11 can be closed. The other end of each air supply pipe 11 can open into a commonly used air supply pipe through which air can be pumped into the air supply pipes 11.

The air supply pipes 11 can be arranged below pressure reduction elements 12. The air supply pipes 11 can be arranged laterally adjacent to the underside of the lowest pressure reduction elements 12.

The pressure reduction elements 12 of one plane can be arranged offset and/or overlapping to pressure reduction elements 12 that are located in an adjacent plane. Each pressure reduction element 12 can extend from one side wall 3 to an opposite side wall 3 and thus over the entire depth of the breeding container 1. The ends of each pressure reduction element 12 can be attached to the two opposite side walls 3.

Pressure reduction elements 12 and/or air supply pipes 11 can be arranged such that one or more shafts 13 remain within the breeding container through which feed can fall downward onto the bottom 2.

At the top side, there can be at least one pipe 14 for the supply of feed above the pressure reduction elements 12, via which wet feed can be pumped into the breeding container 1. From the feed supply pipe 14, pipe stubs 15 can branch off, for example perpendicularly, and be directed downward. The pipe stubs 15 can end above the shafts 13. Wet feed can then fall through the one or more shafts 13 to be able to reliably reach the bottom of the breeding container 1 completely. The feed supply pipe 14 can comprise a valve 16 to be able to open and close the feed supply pipe 14. The feed supply pipe 14 can be attached to one or more rods that can be attached to one or two opposite side walls 3. The one or more rods can be arranged perpendicular to the one or more feed supply pipes 14. Exactly one feed supply pipe 14 can suffice, which can preferably run centrally between two side walls 3 and parallel to the two side walls 3.

Above the ramp 7, there can be a slope 17 that runs in the same way as the ramp 7. The slope can be closed or only be formed by oblique areas on side walls 3. The course of the top side can correspond to the underside such that two breeding containers 1 can be stacked on top of each other. The top side can be limited to the top side of the side walls 3, so that a single breeding container 1 present as a module can initially be completely openly accessible upward. The bottom 2 of the upper breeding container 1 can then close the top side of the lower breeding container 1. For the uppermost breeding container 1, a separate cover can then be provided.

FIG. 2 shows a side view of the breeding container 1 from FIG. 1. At the top side of a side wall 3, there can be an air outlet opening 18 that can be protected by louvers. Behind the air outlet opening 18, a fan or a blower can be arranged. At the underside, there can be an air inlet opening 19 via which air can be pumped into the one or more air supply pipes 11. At the opposite side wall 3, there can also be an air outlet opening 19. The air outlet openings can be arranged diagonally opposite each other.

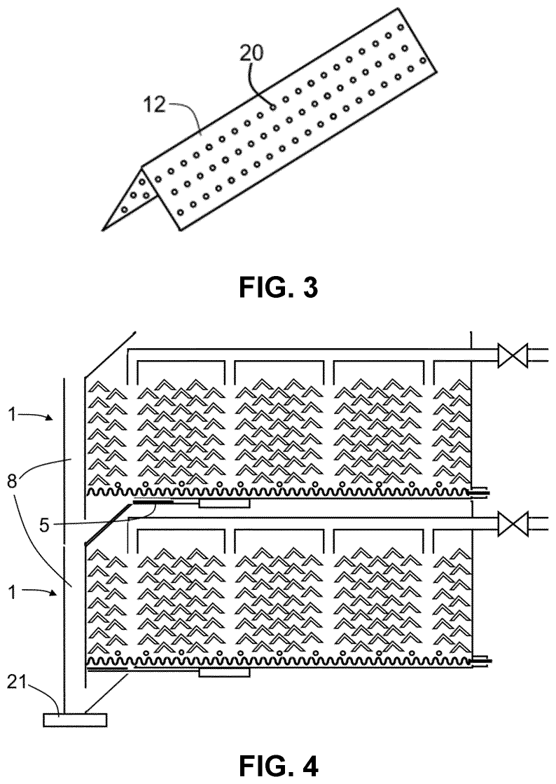

In FIG. 3, a pressure reduction element 12 is shown. This can have, for example, three rows of holes 20 on each side. A row of holes 20 can be arranged offset relative to an adjacent row of holes to optimize the desired effect. The diameter of each hole 20 can be smaller than 10 mm or smaller than 5 mm.

FIG. 4 shows two stacked breeding containers 1. The removal shafts 8 of the two breeding containers 1 join together to form one shaft. Only at the underside of the lowermost removal shaft 8 can a separation device 21 then be provided that can separate feces from insect larvae. If, for example, the uppermost slider 5 has been moved into the opening position as shown, then the contents of the uppermost breeding container 1 can reach the separation device 21 through the removal shaft 8 of the breeding container 1 located below.

The foregoing invention has been described in accordance with the relevant legal standards, thus the description is exemplary rather than limiting in nature. Variations and modifications to the disclosed embodiment may become apparent to those skilled in the art and fall within the scope of the invention.

1. A device for growing insect larvae with a breeding container, wherein a plurality of pressure reduction elements are present in the breeding container, which are arranged above each other and next to each other, and at least one air supply pipe at a bottom of the breeding container, via which air can be pumped into the breeding container.

2. The device according to claim 1, wherein a pump or a compressor is connected to the breeding container such that air can be pumped into the breeding container via the at least one air supply pipe.

3. The device according to claim 1, wherein at least one air outlet opening is present at a top side of the breeding container.

4. The device according to claim 3, wherein a fan or blower is arranged at the air outlet opening to be able to blow air out of the breeding container through the air outlet opening.

5. The device according to claim 1, wherein a feed supply pipe is present at a top side of the breeding container, via which feed can be pumped into the breeding container.

6. The device according to claim 1, wherein at least one vertically running shaft is present within the breeding container between at least two of the plurality of pressure reduction elements.

7. The device according to claim 6, wherein a feed supply pipe is present at a top side of the breeding container via which feed can be pumped into the breeding container, and a stub of the feed supply pipe ends above the at least one vertically running shaft such that feed can fall from the stub into the at least one vertically running shaft.

8. The device according to claim 1, wherein at least one screw conveyor is arranged between the bottom of the breeding container and the pressure reduction elements, which can convey contents from the breeding container to an outlet opening.

9. The device according to claim 8, wherein the outlet opening can be closed by a closure of the device.

10. The device according to claim 9, wherein adjacent to the outlet opening a ramp is present via which contents can slide down from the breeding container.

11. The device according to claim 1, wherein on an outside of a side wall of the breeding container a removal shaft is present into which contents from a second breeding container of the device can be conveyed.

12. The device according to claim 1, wherein the breeding container is a first breeding container, further including a second breeding chambers stacked on or under the first breeding container.

13. A method for growing insect larvae with a device according to claim 1, wherein first the breeding container of the device is filled with feed for the insect larvae to less than 10 cm and subsequently insect larvae are added, wherein the breeding container is at least 0.5 m high.

14. The method according to claim 13, wherein the feed is a flowable biomass.

15. The method according to claim 13, wherein after the addition of the insect larvae, feed is added periodically until the usable volume of the breeding container has been filled to at least 80%.

16. The method according to claim 15, wherein the periodic addition of feed is stopped and after stopping, the contents in the breeding container are only removed when the contents are dry.

17. The method according to preceding claim 13, wherein insect larvae of the black soldier fly are bred.

18. The method according to claim 13, wherein a combustible gas or natural fertilizer is produced from the feces of the insect larvae.

19. The method according to claim 13, wherein a liquid fuel is produced from the insect larvae.

20. The method according to claim 13, wherein at least parts of the insect larvae are used as feed for animals.
